(19) United States
(12) Patent Application Publication
(10) Pub. No.: US 2006/0247503 A1
(43) Pub. Date: Nov. 2, 2006

(54) Method for predicting a transition to an increased probability of injury

(76) Inventors: Orlando Sellers II, Peoria, IL (US); David P. Will, Peoria, IL (US)

Correspondence Address: CATERPILLAR INC.;100 N.E. ADAMS STREET, PATENT DEPT., PEORIA, IL, 616296490, US

(21) Appl. No.: 11/117,983

(22) Filed: Apr. 29, 2005

(51) Int. Cl. A61B 5/00; A61B 19/00

(52) U.S. Cl. 600/300; 128/897; 128/920

(57) ABSTRACT

A method and system for determining an increased probability of injury to a person. The method and system includes determining at least one factor indicative of a propensity to anatomical failure, determining a probability of an anatomical failure occurring as a function of the at least one factor, the anatomical failure being correlated to an injury, and predicting an increased probability of anatomical failure about to occur as a function of the determined probability, the at least one factor, and a period of time of a condition.

### TECHNICAL FIELD

This invention relates generally to a method and system for determining a condition of an increased probability of injury to a worker and, more particularly, to a method and system for predicting when an increased probability of injury to a worker is likely to occur.

### BACKGROUND

It is common knowledge that a person is typically at an increased risk of being injured when performing work at a job that is new and unfamiliar. As the person gains familiarity with the job duties, the risk of injury is expected to decrease. Thus, a person with the proper skills and experience is expected to, over time, maintain a low risk of being injured on the job.

There are several factors, however, that combine to create a situation in which the same person may experience a sharp increase in the risk of injury in spite of that person's familiarity with the job. Factors such as job complacency from over-familiarity and stress may actually act to increase the person's odds of being injured. The time up to this sharp rise in risk factor may be characterized as the useful life of the worker.

A particular concern involves cumulative exposure to physical stress, more particularly cumulative exposure to specific anatomical physiological stress. This may be compared analogously to failure by fatigue of composite materials, such as from repeated bending and flexing. Over time, a worker may become more prone to injury due to this prolonged exposure.

If the tools are available to determine, i.e., predict, when a rise in risk of injury is about to occur, steps may be taken to alleviate the problem and avoid costly injury.

The present invention is directed to overcoming one or more of the problems as set forth above.

### SUMMARY OF THE INVENTION

In one aspect of the present invention a method for determining an increased probability of injury to a person is disclosed. The method includes determining at least one factor indicative of a propensity to anatomical failure, determining a probability of an anatomical failure occurring as a function of the at least one factor, the anatomical failure being correlated to an injury, and predicting an increased probability of anatomical failure about to occur as a function of the determined probability, the at least one factor, and a period of time of a condition.

In another aspect of the present invention a method for predicting a transition to an increased probability of injury is disclosed. The method includes determining at least one factor including at least one of a gross cumulative physical stress and a specific cumulative anatomical stress, calculating a probability of anatomical failure as a function of the determined exposure, and predicting a transition to an increased probability of injury as a function of the probability of anatomical failure and an elapsed period of time.

In yet another aspect of the present invention a system for predicting a transition to an increased probability of injury to a person associated with a condition is disclosed. The system includes a computer having links to an input and an output. The computer receives information from the input relating to the person and the condition, determines at least one factor based on the information, calculates a probability of an anatomical failure occurring based on the at least one factor, and predicts a transition to an increased probability of injury based on the calculated probability.

### BRIEF DESCRIPTION OF THE DRAWINGS

FIG. 1 is a graph embodying one aspect of the present invention;

FIG. 2 is a graph embodying another aspect of the present invention;

FIG. 3 is a graph embodying another aspect of the present invention;

FIG. 4 is a graph embodying another aspect of the present invention;

FIG. 5 is a graph embodying another aspect of the present invention;

FIG. 6 is a block diagram illustrating a computer suited for use with the present invention; and

FIG. 7 is a flow diagram illustrating a method of the present invention.

### DETAILED DESCRIPTION

Referring to the drawings and the appended claims, a method and system 600 for predicting a transition to an increased probability of injury is disclosed. The present invention is described with exemplary reference to a worker at a job. However, the present invention may find application for a person in general associated with a condition. The condition may be a job, but may also include other conditions, such as a sporting or recreational activity, and any other activity that a person may engage in over a period of time.

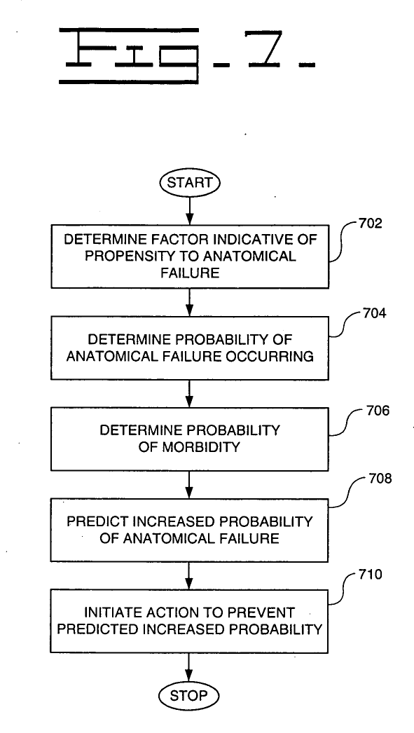

FIG. 6 illustrates a block diagram of a system 600 that may be used with the present invention. A computer 602 is linked to an input 610 and an output 612. The computer 602 may include a central processing unit (CPU) 604, and some form of memory storage, such as RAM 606, ROM 608, and the like. The computer 602 may include other components as well, such as is known in the art.

The input 610 may be any of a variety of devices used for inputting information to a computer. For example, the input 610 may include a keyboard, a mouse, a serial data link, and the like. Multiple combinations of inputs may be used.

The output 612 may be any of a variety of devices used for receiving information from a computer. For example, the output 612 may include a monitor, a printer, a serial data link, and the like. Multiple combinations of outputs may be used.

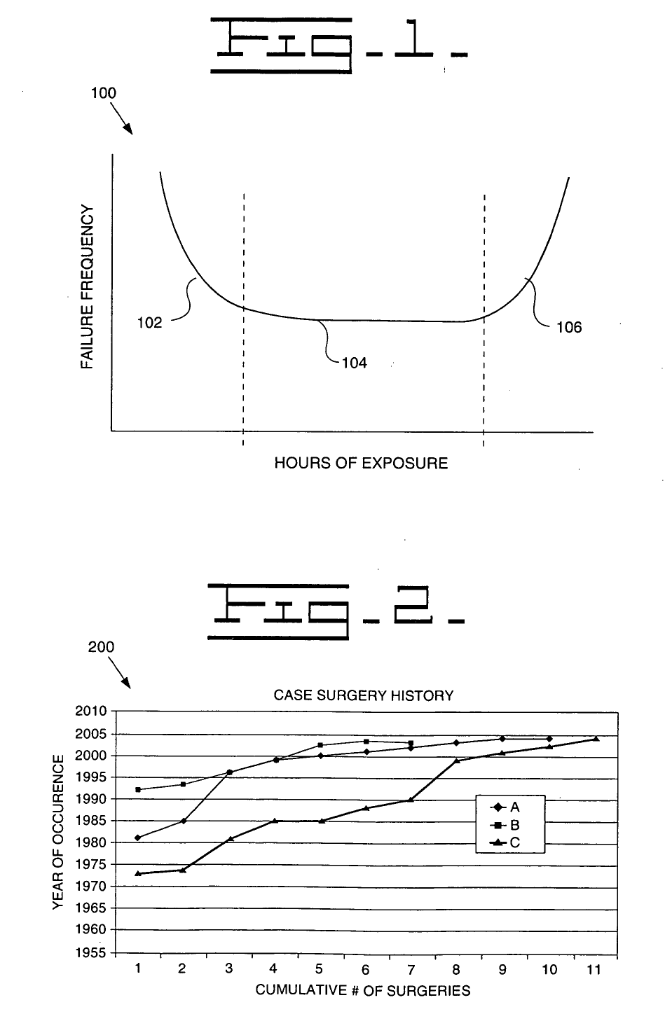

Referring to FIG. 1, a failure frequency graph 100 is shown. More specifically, the failure frequency graph 100 indicates a plot of failure frequency over a period of time. The period of time in the failure frequency graph 100 is depicted in hours, although any unit of time, such as days, weeks, and the like, may be used. The period of time is also depicted as hours of exposure, thus referring to the time that a person is exposed to a condition, e.g., a job. The period of time may be a cumulative period, such as the overall length of time that a person is at a job, for example 10 to 20 years. Alternatively, the period of time may be indicative of a continuous period of time of exposure, such as a worker at a job for a continuous period of time, for example 8 to 10 hours.

The plot of the failure frequency graph is shown in three portions 102,104,106. The first portion 102 begins at a high level of failure frequency and rapidly slopes down to a low level. Typically, this is the expected failure frequency of a person who is new to a condition. For example, a worker that is newly hired to a job may be highly susceptible to injury during an initial learning stage. As the worker becomes familiar with the job, the failure frequency, i.e., the probability of injury, is expected to decrease considerably.

The second portion 104 is indicative of a period of time in which failure frequency is expected to be low. This portion 104 may be characterized as a useful life portion, and may have a time period of, for example, 10 to 30 years, depending upon a number of factors, including the type of condition. For example, if the condition is a job, a more dangerous type of job may have a shorter useful life portion, e.g., about 10 years, than a less dangerous type of job, e.g., about 25 to 30 years.

The third portion 106 indicates a sharp increase in failure frequency. During this period of time, for a variety of reasons as described below, the probability of injury sharply increases. It is desirable to avoid entry into the third portion 106, preferably by anticipating, i.e., predicting, when the third portion 106 is about to occur, and then taking steps to avoid occurrence.

The probability of anatomical failure, i.e., the probability of injury, may be determined as a function of a number of factors, such as gross cumulative physical stress, specific cumulative anatomical stress,.job task labor hours, comorbidity, genetic attributes, mental acuity level, motivation level, engagement level, and the like. The probability of anatomical failure may be determined as a function of any one or a combination of any portion of these factors. In addition, a constant may be used as an additional factor to account for variability in the determination.

The probability of anatomical failure may be determined as a percentage chance relative to other persons engaged in like conditions, or may be determined as a percentage chance relative to a predetermined nominal standard. The probability of anatomical failure may also be determined as a percentage chance of future injury within a given time frame. Other standards of measurement for the probability of anatomical failure may be employed as well.

Gross cumulative physical stress relates to a stress severity index from a whole body perspective. The term “gross” refers to an overall system failure concept, in particular overall stress to the whole body. Gross cumulative physical stress is exemplified as a cumulative trauma that occurs over a long period of time, for example carpal tunnel syndrome.

Gross cumulative physical stress may be categorized as a chronic failure as opposed to an acute failure. An acute failure may be defined as a sudden trauma, in particular on a specific body part. A specific cumulative anatomical stress may fall into the category of an acute failure on a specific body part, such as a lifting injury.

Job task labor hours typically refers to the number of labor hours spent performing a specific task. The probability of anatomical failure determination may factor job task labor hours on a per person basis or as a group. A comparison may then be made either as person to group or group to group to determine if the job task labor hours are within a predetermined norm.

Comorbidity determines how previous failures, i.e., injuries, make new failures more likely. For example, osteoarthritis is a comorbidity condition that makes a person more likely, i.e., susceptible, to be injured. Comorbidity may be other than physical in nature. Examples of comorbidity may include diabetes, hypertension, high cholesterol, and the like.

Genetic attributes refers to basic physical and physiological differences between people, and may include pre-existing genetic abnormalities. For example, a person may be more genetically inclined to adverse skin conditions from exposure to the sun.

Mental acuity level is a measure of how aware a person is during exposure to a condition. The premise is that a less aware person may be more susceptible to injury. Mental acuity level may be genetic, such as a person's mental capacity, or may be a function of a number of factors, such as drinking, long work hours, emotional stress, and the like.

Motivation level is based on the idea that a lesser motivated person may be more prone to injury. Examples include persons with conditions of depression, who do not like their job, who do not like their coworkers, and the like.

Engagement level is a corollary to motivation, and is how much a person is engaged to an activity. Typically, engagement level is higher when a person has a sense of ownership or pride, and likes a job. Higher engagement levels tend to cause a person to function more safely.

The various factors used to determine the probability of anatomical failure, such as those described above, are quantified by some means so that the probability may be determined as a numeric value, such as a percentage. In some cases, individual factors are weighted given their relative importance to the compilation of factors that are used.

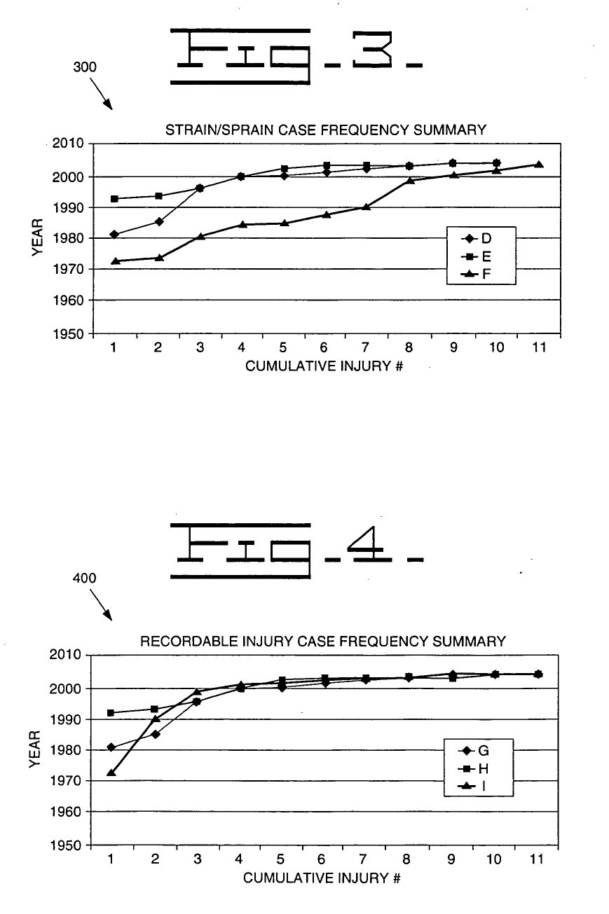

Referring to FIGS. 2-5, a series of graphs to determine a probability of morbidity are shown. The probability of morbidity may be determined as an alternative to the probability of anatomical failure, or may be determined as a supplement, thus performing verification of the probability of anatomical failure determination. Any combination of the graphs may be used, and additional graphs not shown may be used as well.

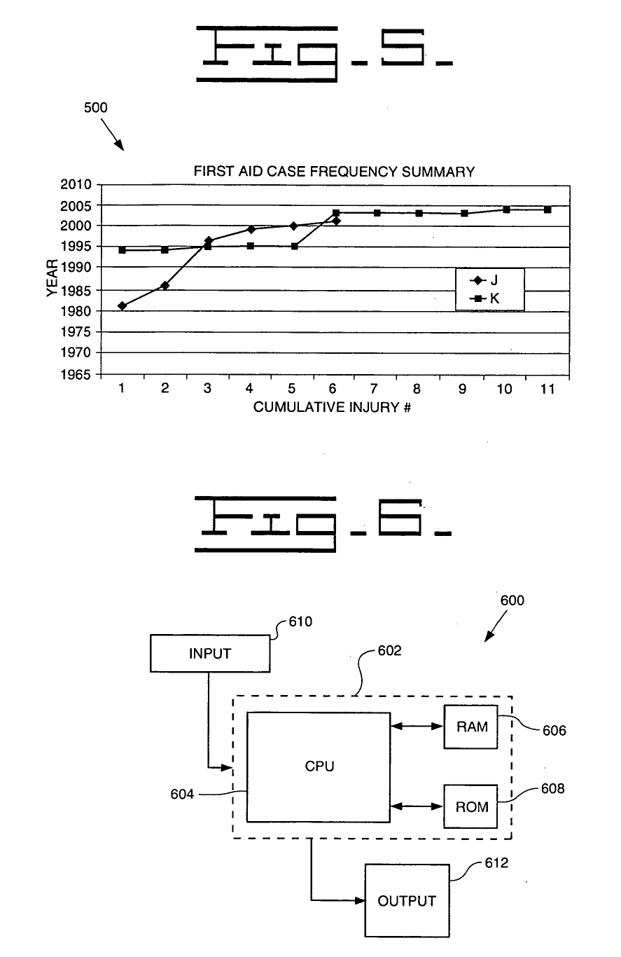

FIG. 2 is a surgery history graph 200. FIG. 3 is a strain/sprain graph 300. FIG. 4 is a recordable injury graph 400. FIG. 5 is a first aid graph. All of the depicted graphs plot year of occurrence as a function of an event history of some type. For example, the surgery history graph of FIG. 2 shows plots of years of occurrence as a function of surgeries performed on persons.

Referring to the plot of subject C on the surgery history graph 200 of FIG. 2 for explanatory purposes, a first surgery is performed on subject C in the year 1973. A second surgery is performed in 1974. A third surgery is performed in 1981. In 1985, two additional surgeries are performed. The next surgery on subject C is in 1988. A seventh surgery is performed in 1990. The next surgery takes place in 1998, with three additional surgeries being performed in 2001, 2002 and 2004, respectively.

A review of the plot of subject C reveals that the frequency of surgeries during a period of time increases as the slope of the plot decreases, i.e., approaches zero. In general terms, the slope of a curve in any of the plots of FIGS. 2-5 may be used as an indicator of a frequency of a corresponding event. In particular, as the slope decreases, the frequency of occurrence of an event increases. Determination of frequency of an event may be made by analyzing the slope directly or by indirect means, such as y=f(x) calculations, and the like. Each of the various frequency determinations may be weighted by some predetermined amount to account for variations in relative importance and significance of each factor.

### INDUSTRIAL APPLICABILITY

Referring to FIG. 7, a flow diagram depicting a method for determining an increased probability of injury to a person is shown. More particularly, the flow diagram of FIG. 7 illustrates a method for predicting a transition to an increased probability of injury.

In a first control block 702, at least one factor is determined which is indicative of a propensity to anatomical failure, i.e., injury. The factors may include any combination of the above described factors, and may include additional factors as well.

In a second control block 704, the probability of an anatomical failure occurring is determined as a function of the above determined factors. The probability may be a probability of anatomical failure, a probability of morbidity, as noted in a third control block 706, or some combination of the two.

In a fourth control block 708, a prediction is made as to an increased probability of anatomical failure being about to occur. The prediction may be made as a function of the determined probability the determined factors, and a period of time of a condition. The prediction may be visualized in the failure frequency graph 100 of FIG. 1 as being, in time, in the second portion 102 of the curve, and predicting an imminent future entry into the third portion 106 of the curve.

In a fifth control block 710, an action may be initiated to prevent entry into the predicted increased probability of anatomical failure. Exemplary actions to take might include providing awareness training to the subject, i.e., person, to attempt to alter one or more factors or, alternatively, reassignment of the person to a different condition, e.g., job, before increased injury occurs.

The present invention, when used in the work environment by employers, may offer the benefit of providing a metric to control costs for health care of workers, and may serve to increase labor efficiency and productivity. Another benefit may be to provide a standard of measure of work expectations, and thus help identify possible issues of employee misuse relative to the reporting of injuries.

Other aspects can be obtained from a study of the drawings, the disclosure, and the appended claims.

What is claimed is:

1. A method for determining an increased probability of injury to a person, comprising the steps of:
determining at least one factor indicative of a propensity to anatomical failure;
determining a probability of an anatomical failure occurring as a function of the at least one factor, the anatomical failure being correlated to an injury; and
predicting an increased probability of anatomical failure about to occur as a function of the determined probability, the at least one factor, and a period of time of a condition.

2. A method, as set forth in claim 1, wherein a condition includes a job, and a period of time of a condition includes a time of exposure to the job.

3. A method, as set forth in claim 1, further including the step of initiating an action to prevent the predicted increased probability of anatomical failure.

4. A method, as set forth in claim 1, wherein determining at least one factor includes the step of determining at least one of a gross cumulative physical stress and a specific cumulative anatomical stress.

5. A method, as set forth in claim 4, wherein determining at least one factor includes the step of determining at least one of a job task period of time, a comorbidity, a genetic attribute, a mental acuity level, a motivation level, and an engagement level.

6. A method, as set forth in claim 1, further including the step of determining a probability of morbidity as a function of at least one frequency factor.

7. A method, as set forth in claim 6, wherein the at least one frequency factor includes at least one of a frequency of first aid cases, a frequency of recordable injury cases, a frequency of surgical interventions, a frequency of strains, and a frequency of sprains.

8. A method for predicting a transition to an increased probability of injury, comprising the steps of:
determining at least one factor including at least one of a gross cumulative physical stress and a specific cumulative anatomical stress;
calculating a probability of anatomical failure as a function of the determined exposure; and
predicting a transition to an increased probability of injury as a function of the probability of anatomical failure and an elapsed period of time.

9. A method, as set forth in claim 8, wherein determining at least one factor further includes determining at least one factor including at least one of a job task period of time, a comorbidity, a genetic attribute, a mental acuity level, a motivation level, and an engagement level.

10. A method, as set forth in claim 8, wherein calculating a probability of anatomical failure includes the step of calculating a percent chance of an anatomical failure relative to a standard.

11. A method, as set forth in claim 10, wherein calculating a percent chance of anatomical failure relative to a standard includes the step of calculating a percent chance of anatomical failure of a person relative to a group of other persons.

12. A method, as set forth in claim 8, wherein calculating a probability of anatomical failure includes the step of calculating a percent chance of an anatomical failure occurring in a given time frame.

13. A system for predicting a transition to an increased probability of injury to a person associated with a condition, comprising:
a computer having links to an input and an output;
wherein the computer receives information from the input relating to the person and the condition, determines at least one factor based on the information, calculates a probability of an anatomical failure occurring based on the at least one factor, and predicts a transition to an increased probability of injury based on the calculated probability.

14. A system, as set forth in claim 13, wherein the computer delivers the predicted transition to the output.

15. A system, as set forth in claim 13, wherein the person is a worker and the condition is a job.

16. A system, as set forth in claim 13, wherein the at least one factor includes at least one of a gross cumulative physical stress, a specific cumulative anatomical stress, a job task period of time, a comorbidity, a genetic attribute, a mental acuity level, a motivation level, and an engagement level.

17. A system, as set forth in claim 13, wherein the at least one factor includes at least one of a frequency of first aid cases, a frequency of recordable injury cases, a frequency of surgical interventions, a frequency of strains, and a frequency of sprains.

18. A system, as set forth in claim 17, wherein each of the at least one factor is assigned a weight.

19. A system, as set forth in claim 17, wherein the computer calculates a probability of morbidity based on the at least one factor.
